(19) United States
(12) Patent Application Publication
(10) Pub. No.: US 2006/0020175 A1
(43) Pub. Date: Jan. 26, 2006

(54) Method for managing deselection of medicine

(75) Inventors: Lisa Berry, Ambler, PA (US); Jerry B. Hansen, Blue Bell, PA (US); Robert Hasselman, North Sydney (AU); Alexandra Langendorfer, Philadelphia, PA (US); Stephanie Levy, Melrose Park, PA (US)

Correspondence Address: CONNOLLY BOVE LODGE & HUTZ, LLP, P O BOX 2207, WILMINGTON, DE, 19899, US

(73) Assignees: McNeil-PPC, Inc., Skillman, NJ (US); Merck & Co., Inc., Rahway, NJ (US)

(21) Appl. No.: 11/185,385

(22) Filed: Jul. 20, 2005

Related U.S. Application Data
(60) Provisional application No. 60/590,261, filed on Jul. 21, 2004.

(51) Int. Cl. A61B 5/00

(52) U.S. Cl. 600/300; 705/3

(57) ABSTRACT

A method for managing the treatment regimen of a human patient using an over-the-counter (OTC) medicine is provided. The method comprises providing to the patient an interactive therapy managing system for de-selecting himself from the treatment regimen. The interactive therapy managing system may comprise, for example, an in person contact, or an electronically enabled element, such as an automated voice response system, a text-messaging system, an email system, a touch-tone telephone menu system, and a website.

This application claims benefit to U.S. Provisional Patent Application 60/590,261 filed Jul. 21, 2004 which is incorporated by reference herein in its entirety.

### BACKGROUND OF THE INVENTION

The present invention relates to the management of treatment regimens of over-the-counter (OTC) medicines (sometimes referred to as non-prescription medicines), and in particular the management of a patient's de-selection of an OTC medicine, that is, a patient's decision to modify his treatment regimen in some way or stop taking the medicine altogether.

OTC medicines are conveniently used by individual patients to treat a wide variety of conditions such as headaches and heartburn. However, unlike such symptomatic conditions, asymptomatic or chronic conditions like hypertension, high cholesterol, or osteoporosis pose a risk of inappropriate administration. Asymptomatic conditions seldom exhibit symptoms that indicate if, when, or how much medication to take. Compliance with the recommended treatment regimen is particularly low among patients with asymptomatic conditions, even when under physician supervision. Accordingly, patients need to consider more complex information to determine whether a medicine for treating an asymptomatic or chronic condition is right for them, how to use the medicine appropriately, and when to stop. Proper guidance to consumers is critical in transitioning a medicine from prescription to over-the-counter use. The United States Food and Drug Administration (FDA) requires suitable safeguards to be in place to assure the product is right for the consumer and the consumer is properly instructed in its use before it will allow OTC sale of a medicine for treatment of asymptomatic or chronic conditions. Additionally, chronic preventative treatment for certain symptomatic conditions, such as asthma, may present similar compliance issues during prolonged asymptomatic periods due to the preventative regimen.

WO 02/05187 A1 relates a method for reducing consumer errors in, first, selecting a medicine for treatment of an asymptomatic or chronic condition and, second, encouraging appropriate long-term use of the medicine. The first step of the method includes a stepwise guide to understanding label information that leads the consumer through the criteria for determining whether use of this drug is appropriate. The next step involves directing the consumer to package inserts, including an informational booklet and visual media such as a videotape, further explaining the label information. Yet another step involves communication of incentives to maintain long term usage, including incentives to encourage communication with a trained product specialist. For eligible consumers, the product specialist gives answers to questions about proper use of the product. For ineligible consumers, the product specialist advises the consumer to discuss the product and the condition with a doctor and facilitates a full purchase price refund.

Although the methods of WO 02/05187 A1 are concerned with a patient's initial decision to use a medicine (which may be a decision not to use the medicine in the first place) and continue its long-term use, WO 02/05187 A1 does not address a patient's decision to de-select the medicine after a period of use. The concept of encouraging de-selection of a chronically administered medicine, or for that matter most consumer products is an unexpected one. Most marketers of products are concerned exclusively with encouraging consumers to begin and maintain use of their products, not to stop using them. However, in the case of OTC medicines, de-selection, when appropriate, is also a critical step. De-selection may be appropriate for example, if the patient has any concurrent health conditions for which the OTC medicine is contra-indicated, is concomitantly using any medications known to interact with the OTC medicine, is not experiencing the desired therapeutic response to the treatment regimen which may not be apparent to the patient who has an asymptomatic condition, or is experiencing symptoms of known adverse effects of the OTC medicine. One of the benefits of this invention is that it addresses the difficulty associated with making a patient aware that he may need to de-select the medicine, particularly where the patient does not have outwardly recognizable symptoms because the condition being treated is asymptomatic.

### SUMMARY OF THE INVENTION

Accordingly, the invention provides a method for managing the treatment regimen of a human patient using an over-the-counter (OTC) medicine comprising providing to the patient an interactive therapy managing system for de-selecting himself from the treatment regimen. In one embodiment, the method further comprises prompting the patient for information; receiving information from the patient, including information about said treatment regimen; comparing said information with disqualifying criteria for said medicine; and in the event the patient meets said disqualifying criteria, recommending to the patient that he modify the treatment regimen.

In another embodiment, the invention provides a method for guided self-management and promoting appropriate de-selection of an over-the-counter (OTC) medicine being self administered by an individual consumer, comprising: receiving information from said consumer; comparing said information with disqualifying criteria for said medicine; and in the event said consumer meets said disqualifying criteria, recommending to said consumer that he adjust use of said medicine, cease use of said medicine, or contact a physician.

### DETAILED DESCRIPTION OF THE INVENTION

As used herein, the terms “patient” and “consumer” are used interchangeably. Both refer to a human user of an OTC medicine.

The present methods are useful with a variety of OTC medicines used to treat a variety of health conditions. In one embodiment of the invention, the health condition is an asymptomatic health condition. Examples of this include hypercholesterolemia, osteoporosis, hypertension, and calcium deficiency. The health condition may also be chronic. In another embodiment of the invention, the health condition may be symptomatic, while the treatment is preventative, and the preventative treatment regimen is chronic. One example of a chronic preventative treatment for a symptomatic condition is the use of leukotriene receptor antagonists such as montelukast for the prophylaxis and chronic treatment of asthma.

One application of this invention is in the modification of lipid levels, including but not limited to total cholesterol, low density lipoprotein (LDL) cholesterol, high density lipoprotein (HDL) cholesterol, and triglycerides. Consumers are aware that high LDL cholesterol is a health risk and many of these individuals are interested in a nonprescription cholesterol-reducing product to maintain healthy cholesterol levels. Lovastatin (10 mg to 80 mg), currently marketed as a prescription drug, is an effective cholesterol-lowering agent. It is sold in the United States by Merck & Co., Inc. under the MEVACOR® label.

Other examples of cholesterol lowering agents include but are not limited to statins in their lactonized or dihydroxy open acid forms and pharmaceutically acceptable salts and esters thereof, such as simvastatin (US tradename ZOCOR®, see U.S. Pat. No. 4,444,784); dihydroxy open-acid simvastatin, particularly the ammonium or calcium salts thereof; pravastatin, particularly the sodium salt thereof (US tradename PRAVACHOL®, see U.S. Pat. No. 4,346,227); fluvastatin, particularly the sodium salt thereof (US tradename LESCOL®, see U.S. Pat. No. 5,354,772); atorvastatin, particularly the calcium salt thereof (US tradename LIPITOR®, see U.S. Pat. No. 5,273,995); rosuvastatin, particularly the calcium salt thereof (US tradename CRESTOR®, see U.S. Pat. No. Re 37314); and pitavastatin also referred to as NK-104 (see PCT international publication number WO 97/23200). Ezetimibe (US tradename ZETIA®, see US Patent No. Re. 37721) is in a class of lipid-lowering compounds that selectively inhibits the intestinal absorption of cholesterol and related phytosterols.

Additional examples of cholesterol modifying agents include those that raise HDL cholesterol levels, such as niacin in immediate-release and time-controlled release forms, and cholesteryl ester transfer protein (CETP) inhibitors such as torcetrapid and JTT-705, which are currently in development. Combinations of cholesterol modifying agents combined in a single dosage unit or in a kit providing each agent in a separate dosage unit, may also be used for cholesterol level modification. Examples include but are not limited to the combination of two or more of an HMG-CoA reductase inhibitor, a cholesterol absorption inhibitor, a CETP inhibitor and niacin. Specific examples of combinations include lovastatin or simvastatin with ezetimibe, and lovastatin or simvastatin with niacin. Any cholesterol modifying agents that are approved for non-prescription use would be suitable for use in this invention.

The methods are applicable to a patient who is self-administering an OTC medicine. After having taken the medicine for some time period, the patient provides information, preferably to an interactive therapy managing system, that allows him to decide whether de-selection of the treatment regimen is appropriate.

In one embodiment, the method is one for managing the treatment regimen of a human patient using an OTC medicine, and comprises providing to the patient an interactive therapy managing system for de-selecting himself from the treatment regimen.

In another embodiment, the method is one for guided self-management and promoting appropriate de-selection of an OTC medicine being self administered by an individual consumer. The method comprises receiving information from said consumer; comparing said information with disqualifying criteria for said medicine; and in the event said consumer meets said disqualifying criteria, recommending to said consumer that he adjust use of said medicine, cease use of said medicine, or contact a physician.

The interactive therapy managing system comprises a personal or written or electronic source with which the patient may communicate about the OTC medicine he is taking. Particularly with the treatment of an asymptomatic or chronic condition, the patient must receive appropriate information to assist him in making an informed choice about whether to discontinue or modify use of the medicine. Moreover, on a day-to-day basis, the use of an OTC medication for treating an asymptomatic condition may or may not make the patient feel any different. It is important that the patient have access to information and guidance on whether to modify or discontinue his treatment regimen. An added advantage of the method of the present invention is that it may improve patient compliance with the treatment regimen.

The exchange of information between the patient and the interactive therapy managing system may take place without assistance in a drugstore or other retail outlet, or in the patient's home, or other non-retail location. The interactive therapy managing system may comprise, for example, contact with a person such as a trained professional via an in person contact, i.e., contact with a trained product specialist having access to specialized information about the medicine, or a telephone contact with such a person, or a voice or e-mail exchange, or text message exchange with such a person. Alternatively, it may comprise a written aid such as a tear pad or other printed material located at the retail shelf, or special instructions incorporated into the packaging of the product, or a printed newsletter mailed directly to the patient's address. Such written material may comprise textual and/or graphic information. As used herein, the written aid may include any pre-printed or pre-recorded media, such as printed paper or other printed media, compact disc, audio cassette, or other audio recordings, or video disc, video cassette, or other video recordings. In any case, the written aid guides the patient through a decision making process, based on information known to the patient about his particular health condition and/or test result. In yet another embodiment, the interactive therapy managing system may comprise an electronically enabled element such as an automated voice response system, touch-tone telephone menu system, or a website. In certain embodiments, access to the interactive therapy managing system is enabled by an electronic device, for example, a computer, cable television, a cellular telephone, touch-tone telephone, or a personal digital assistant or pocket personal computer. In one preferred embodiment, the interactive therapy managing system is completely automated, with substantially no element of contact between the patient and a person, e.g. a professional. In another preferred embodiment, the interactive therapy managing system may be accessed remotely from any location convenient for the patient. In another preferred embodiment, the type of information prompted for and received by the interactive therapy managing system, and the response of the system to a particular set of answers by the patient will be the same for every patient taking a particular OTC medication, based on a standard set of criteria incorporated into the system.

The interactive therapy managing system prompts for and receives information from the patient. The information received from the patient is relevant to the decision to continue or discontinue the treatment regimen. In certain embodiments, relevant information includes a test result which can be used to assess the success of the treatment regimen in achieving the therapeutic objective. For example, if the over-the-counter medicine is a cholesterol-lowering agent, the test result may include blood cholesterol or triglyceride levels. If the over-the-counter medicine is an antihypertensive, the test result may include one or more blood pressure measurements. If the over-the-counter medicine is a treatment for osteoporosis, the test result may include a bone density measurement or a biochemical marker. The information prompted for and received from the patient may further include relevant health information such as medical history, current health conditions, medication history, current medications being used by the patient concomitantly with the OTC medicine with which treatment is being managed by the method of the present invention. The information prompted for and received from the patient may further include test results related to known adverse effects of the OTC medicine. For example, for certain osteoporosis treatments, the test result may include a creatinine clearance. Additional relevant information collected by the interactive therapy managing system may include parameters such as age, weight, and gender of the patient, the length of time the patient has been taking the medicine, actual compliance performance of the patient with the treatment regimen, alcohol consumption, and smoking history.

The interactive therapy managing system has access to or incorporates instructions based upon the disqualifying criteria for use of the OTC medicine. Disqualifying criteria are specific for the particular OTC medicine being used, and include concurrent health conditions for which the OTC medicine is contra-indicated, concomitant use of medications known to interact with the OTC medicine, lack of therapeutic response to the treatment regimen, a change in severity of the condition for which the medication is being taken and experience of symptoms of known adverse effects of the OTC medicine. These can be found in the professional labeling for each particular medicine, which is published in the Physician's Desk Reference. For example, in the case of certain cholesterol lowering medicines, contraindications include hypersensitivity to any component of the medication (active ingredient or inactive ingredients in the dosage form); active liver disease, or unexplained persistent elevations of serum transaminases; pregnancy and lactation. Concomitant medications to be avoided with certain cholesterol lowering medicines, include those known to be associated with myopathy, e.g. gemfibrizol or other fibrates, and lipid lowering doses (>1 g/day) of niacin; inhibitors of the cytochrom P450 isoform 3A4 metabolic pathway, e.g. cyclosporine, the azole antifungals itraconazole and ketoconasole, the macrolide antibiotics erythromycin and clarithromycin, HIV protease inhibitors, the antidepressant nefazodone, and large quantities of grapefruit juice (>32 oz/day). Potential adverse effects of certain cholesterol lowering medicines include myopathy (muscle pain or weakness); liver dysfunction, as indicated by persistent increases in serum transaminases. The interactive therapy managing system would have access to or incorporates instructions based upon information on any and all contraindications, drug interactions, and potential adverse effects with the OTC medication. In the case of cholesterol lowering medicines, some of these are CYP3A4 inhibitors, gemfibrozil, niacin, and cyclosporin.

When the interactive therapy managing system comprises a telephone contact or website, the telephone number or domain name are preferably selected to designate the OTC medicine or a condition it treats in order to promote recognition by the patient and insure that inquiries are properly directed to a product specialist fully conversant with the medicine. Thus for example, a patient who dials a telephone number such as 1-800-MEVACOR or types in a domain name such as mevacor.com will be connected with the interactive therapy managing system, which may include an option for contact with a product specialist familiar with MEVACOR® and its disqualifying criteria, or may be operated substantially free of human contact. While the phone number and/or domain name preferably have a sound or connotation corresponding to the medicine in question, any number or name that assures patient contact with a product specialist familiar with the medicine can be used in accordance with this invention.

Unlike typical toll-free phone numbers or e-mail addresses that merely receive consumer input or complaints, the interactive therapy managing system contemplated by this invention is interactive. A person, or electronically enabled element, or a combination of the two provides advice and information about use and de-selection of the medication.

In one embodiment of the invention, the patient is prompted for information. Information is received by the interactive therapy managing system, including information about the treatment regimen of the patient. The information received from the patient is compared with the disqualifying criteria for the medicine, and in the event the patient meets the disqualifying criteria, a recommendation is made to the patient that he modify the treatment regimen. In one particular embodiment, in which the system comprises a written aid, the written aid may incorporate alternative recommendations in anticipation of all possible patient answers. For example, the written aid may begin with a question such as, “did you obtain a (test result)?”, coupled with a conditional recommendation such as, “if not, please obtain a test result. Here are some ways to obtain the test result . . . ”. And may continue with a question about the test result, such as, “was the result within (desired range)?”, coupled with a conditional recommendation such as, “if not, see your doctor,” and “if yes, keep taking the medication, and re-test after (time interval).” Modification may comprise merely patient self-assessment of their compliance with the treatment regimen and re-testing after a period of improved compliance, or it may comprise adjusting the treatment regimen, ceasing use of the medicine, or contacting a physician. Such modification may comprise two or more of the foregoing actions in combination. When a patient is instructed to contact a physician, the intended outcome of this may include seeking more aggressive treatment in order to achieve the targeted result of treatment.

As described above, the information provided by the patient may include a test result. The patient himself, a testing agent, or a physician may provide such test result to the interactive therapy managing system. In connection with this, the therapy managing system may recommend alternatives to the patient about how and where to obtain a test result. Test results include, for example, bone density, blood pressure, and serum cholesterol levels. Among other things, the test result may indicate that the medication is ineffective, or has improved the condition, but not to the desired level, i.e., the test result is not within the desired target range after a specified duration of treatment.

In one embodiment, an incentive is offered to the patient to encourage information sharing or communication with the interactive therapy managing system after he has been taking the medicine for period of time, followed by adjustment of use of said medicine, ceasing use of said medicine, or contacting of a physician. One example of such an incentive is at least a partial rebate of a purchase price of said OTC medicine.

In another embodiment, a high value incentive that is not described on, or in, the materials in the initial carton containing the medicine is offered. It has been found that such high value incentives are best communicated in a separate incentive coupon. The high value incentives are typically items having considerable appeal to consumers, while having a manageable cost to the supplier of the medicine. One example of such an incentive would be a free blood test or other analytical procedure related to the asymptomatic/chronic condition being treated with the medication. The test results would be used to determine whether the patient should modify his treatment regimen, including stopping use of the medicine altogether. A certificate for such a test can be transmitted to the consumer if deemed appropriate by the product specialist.

What is claimed is:

1. A method for managing the treatment regimen of a human patient using an over-the-counter (OTC) medicine comprising providing to the patient an interactive therapy managing system for de-selecting himself from the treatment regimen.

2. The method of claim 1, wherein said medicine is used to treat an asymptomatic health condition.

3. The method of claim 2, wherein said asymptomatic health condition is selected from the group consisting of hypercholesterolemia, osteoporosis, hypertension, and calcium deficiency.

4. The method of claim 2, wherein said health condition is chronic.

5. The method of claim 1, wherein said interactive therapy managing system comprises an electronically enabled element.

6. The method of claim 5, wherein said electronically enabled element comprises a feature selected from the group consisting of an automated voice response system, a text-messaging system, an email system, a touch-tone telephone menu system, and a website.

7. The method of claim 6, wherein the method further comprises:
prompting the patient for information
receiving information from the patient;
comparing said information with disqualifying criteria for said medicine; and
in the event the patient meets said disqualifying criteria, recommending to the patient that he modify the treatment regimen.

8. The method of claim 7, wherein said modification step is selected from the group consisting of patient self-assessment of compliance with the treatment regimen, adjusting the treatment regimen, ceasing use of said medicine, contacting a physician, and combinations thereof.

9. The method of claim 7, wherein said information provided by said patient includes a test result.

10. The method of claim 9, wherein said method further comprises recommending alternatives to the patient about how to obtain said test result.

11. The method of claim 9, wherein said test result is communicated by the patient to said interactive therapy managing system.

12. The method of claim 9, wherein said test result is communicated on behalf of said individual consumer to said interactive therapy managing system by a testing agent.

13. The method of claim 7, wherein said information provided by said patient includes one or more items selected from the group consisting of information about said treatment regimen, information about concomitant use of additional medicines, information about the patient's health conditions other than the one being treated by the regimen, information about health symptoms occurring since beginning the regimen.

14. The method of claim 7, wherein said disqualifying criteria is selected from the group consisting of concurrent health conditions for which the OTC medicine is contra-indicated, concomitant use of medications known to interact with the OTC medicine, and experience of symptoms of known adverse effects of the OTC medicine.

15. A method for guided self-management and promoting appropriate de-selection of an over-the-counter (OTC) medicine being self administered by an individual consumer, comprising:
receiving information from said consumer;
comparing said information with disqualifying criteria for said medicine; and
in the event said consumer meets said disqualifying criteria, recommending to said consumer that he adjust use of said medicine, cease use of said medicine, or contact a physician.

16. The method of claim 15, wherein said information provided by said consumer includes a test result.

17. The method of claim 16, wherein said test result is selected from cholesterol level, bone density, blood pressure, and serum calcium levels.

18. The method of claim 15, wherein said information is received by an interactive therapy managing system.

19. The method of claim 18, wherein said interactive therapy managing system comprises an element selected from the group consisting of an in person contact, a telephone contact, an automated voice response system, and a website.

20. The method of claim 18, wherein said interactive therapy managing system also recommends alternatives to said individual consumer about how to obtain said test result.

21. The method of claim 18, wherein said interactive therapy managing system comprises an electronically enabled element selected from the group consisting of an automated voice response system, touch-tone telephone menu system, or a website.

22. The method of claim 18, wherein said interactive therapy managing system comprises a written aid, and said written aid guides the patient through a decision making process.

23. The method of claim 22, wherein said written aid incorporates questions coupled with alternative recommendations in anticipation of all possible patient answers to the questions.

24. The method of claim 18, wherein said test result is communicated by said consumer to said interactive therapy managing system.

25. The method of claim 18, wherein said test result is communicated on behalf of said individual consumer to said interactive therapy managing system by a testing agent.

26. The method of claim 15, further comprising offering to said individual consumer an incentive to induce adjustment of use of said medicine, ceasing use of said medicine, or contacting of a physician.

27. The method of claim 26, wherein said incentive is at least a partial rebate of a purchase price of said OTC medicine for said consumer.
